(19) United States
(12) Patent Application Publication
(10) Pub. No.: US 2007/0072253 A1
(43) Pub. Date: Mar. 29, 2007

(54) Method of Determining Standard Activity Value of Nattokinase

(76) Inventors: Chih Yi Lu, Bade City (TW); Jiunn-Ming Jeng, Bade City (TW)

Correspondence Address: HDSL, 4331 STEVENS BATTLE LANE, FAIRFAX, VA, 22033, US

(21) Appl. No.: 11/534,634

(22) Filed: Sep. 22, 2006

(30) Foreign Application Priority Data
Sep. 23, 2005 (TW) 094132962

(51) Int. Cl. C12Q 1/48

(52) U.S. Cl. 435/15

(57) ABSTRACT

A method of determining a standard activity value of Nattokinase includes preparing a standard curve with respect to a plurality of Nattokinases having predetermined activity values by calculating corresponding dilution multiples using a standard essay, obtaining a sample curve with respect to an indeterminate Nattokinase by diluting the indeterminate Nattokinase with a plurality sample dilution multiples to calculate corresponding activity values using the standard essay, and determining a standard activity of the indeterminate Nattokinase by comparing the standard curve and the sample curve in a diagram to obtain an intersection.

### BACKGROUND OF THE INVENTION

The present invention relates to a method of determining a standard activity value of Nattokinase, and particularly to a method which can accurately determine a standard activity value for Nattokinase with unknown activity levels.

### 2. Description of the Related Art

Research has shown Nattokinase to support healthy coagulation of blood within normal levels and enhance fibrinolytic activity. Nattokinase is an enzyme isolated from Natto, a traditional Japanese fermented soy food. More recently, both clinical and non-clinical studies have demonstrated that Nattokinase supports heart health and promotes healthy circulation. Nattokinase help to keep already healthy levels of blood clotting factors within a normal range. As such, lots of biotechnology companies are aggressively invested to supply for the increasing requirements. However, the quality of Nattokinase product is a critical issue. One approach is by determining an activity value of Nattokinase.

Japan Natto Association has announced a method for determining a standard activity value of Nattokinase (called “standard method” hereinafter), which includes comparing a Nattokinase sample to a dummy sample by using the following equation to obtain a relation between the activity value per gram (FU/g) and the dilution multiple (D).
FU/g=(A.sub.τ−A.sub.B)/0.01.times.1/60.times.1/0.1.times.D.
where the A.sub.τ and the A.sub.B are absorbances of the Nattokinase sample and the dummy sample respectively measured at a wavelength of 275 nm.

However, the detail procedures of the standard method is not public so that every one who wants to determine a standard activity value of Nattokinase has to pay for using the standard method as the only way to verify his Nattokinase product by Japan Natto Assocation. Even having the equation of the “standard method” in hand, a standard activity value of Nattokinase still can not be determined. By testing a 4000 FU Nattokinase, as shown in FIG. 1, several dilution multiples are used to obtain the curve. According to the curve in FIG. 1, a dilution multiple about 70 is determined with respect to this 4000 FU Nattokinase. However, as shown in FIG. 2, in another Nattokinase with standard activity value, a dilution multiple about 70 is determined with respect to 8000 FU Nattokinase.

That is, there is no way to determine a standard activity value for an indeterminate Nattokinase by the “standard method” because the corresponding dilution multiple is unknown.

### BRIEF SUMMARY

The present invention is to provide a method of determining a standard activity value of an indeterminate Nattokinase.

According to the present invention, the method of determining a standard activity value of Nattokinase includes the steps of preparing a standard curve with respect to a plurality of Nattokinases having predetermined activity values by calculating corresponding dilution multiples using a standard essay, obtaining a sample curve with respect to an indeterminate Nattokinase by diluting the indeterminate Nattokinase with a plurality sample dilution multiples to calculate corresponding activity values using the standard essay, and determining a standard activity of the indeterminate Nattokinase by comparing the standard curve and the sample curve in a diagram to obtain an intersection.

The standard essay includes comparing a Nattokinase sample to a dummy sample by using an equation of FU/g=(A.sub.τ−A.sub.B)/0.01.times.1/60.times.1/0.1.times.D, where the A.sub.τ and the A.sub.B are absorbances of the Nattokinase sample and the dummy sample respectively measured at a wavelength of 275 nm, to obtain a relation between the activity value per gram FU/g and the dilution multiple D.

Preferably, a multiple difference between the sample dilution multiples is less than 100.

Other objects, advantages and novel features of the invention will become more apparent from the following detailed description when taken in conjunction with the accompanying drawings, in which:

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a diagram showing a curve of 4000 FU Nattokinase to determine the corresponding dilution multiple;

FIG. 2 is a diagram showing a curve of 8000 FU Nattokinase to determine the corresponding dilution multiple;

FIG. 3 is a flowchart showing the steps of the present invention;

FIG. 4 is a diagram showing a standard curve with respect to deferent Nattokinases having predetermined activity values; and

FIG. 4 is a diagram showing a sample curve with respect to an indeterminate Nattokinase having an intersection with the standard curve to determine the standard activity value of the indeterminate Nattokinase.

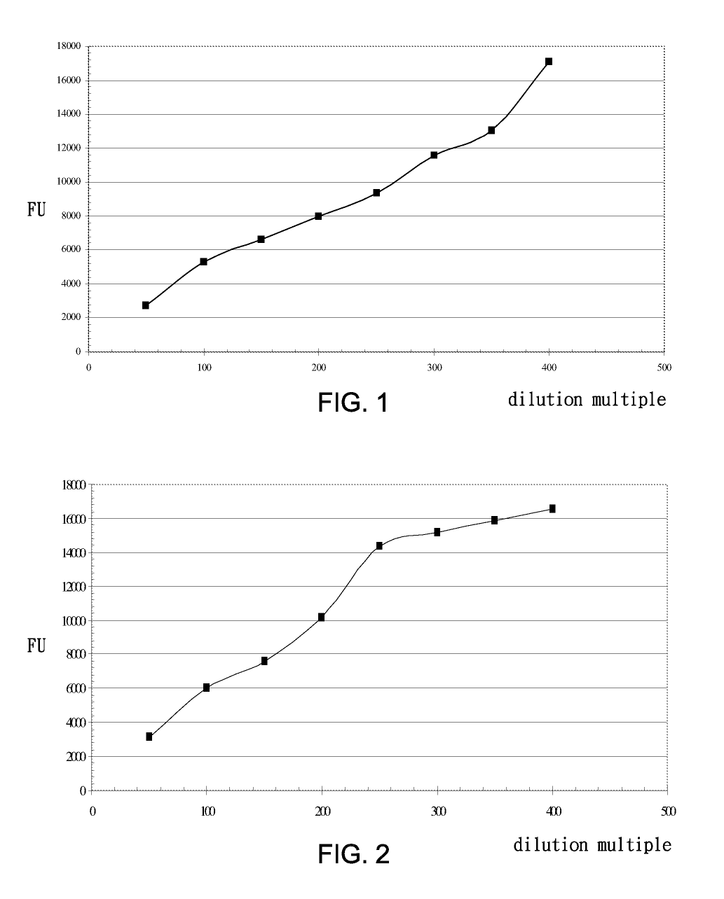

### DETAILED DESCRIPTION

Reference will now be made to the drawings to describe a preferred embodiment of the present method of determining a standard activity value of Nattokinase, in detail.

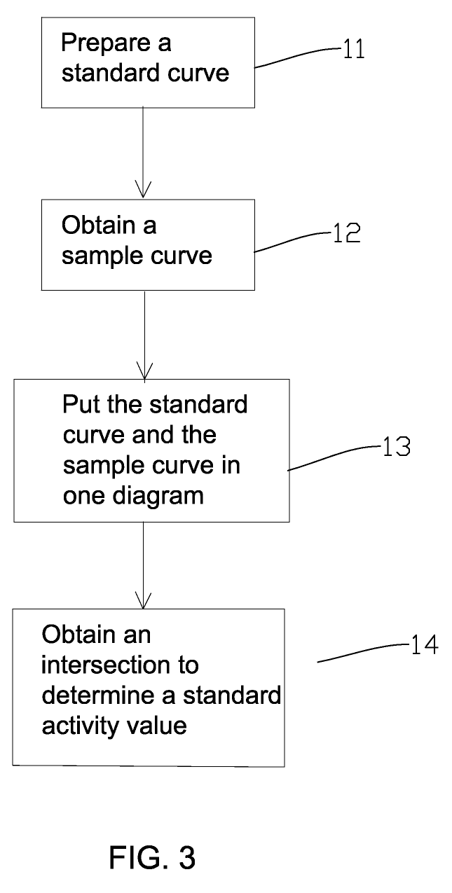

Please refer to FIG. 3, which is a flowchart showing the steps of the method for determining the standard activity value of Nattokinase according to the present invention.

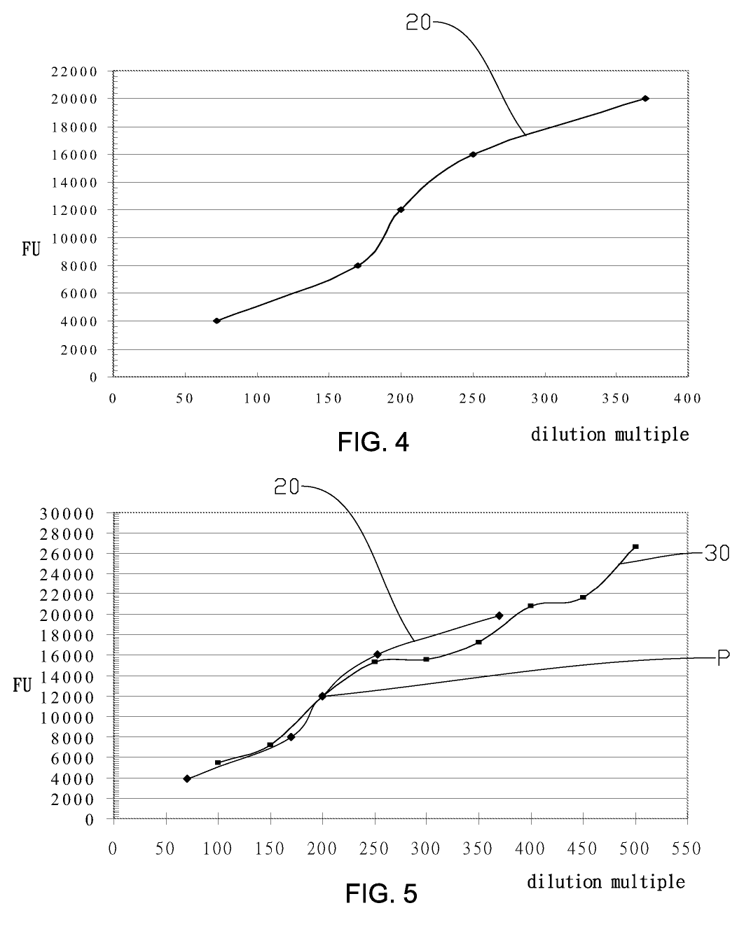

First, in step 11, prepare a standard curve with respect to deferent Nattokinases having predetermined activity values. That is, several Nattokinases with standard activity values determined by Japan Natto Association using the “standard method” are provided. According to the equation of “standard method”, a dilution multiple for each Nattokinase can be calculated by experiment. As such, the standard curve can be depicted. For example, by providing 4000 FU, 8000 FU, 12000 FU, 16000 FU, 20000 FU Nattokinases, the corresponding dilution multiples of about 70, 160, 200, 250, 370 are obtained. Therefore, a standard curve 20 in FIG. 4 is shown. It is noted that providing more Nattokinases with standard activity values determined by the standard method with make the result more accurate in the present invention.

Next, in step 12, obtain a sample curve with respect to an indeterminate Nattokinase. That is, dilute the indeterminate Nattokinase to several samples by various multiples and smaller difference for the dilution multiples of the Nattokinase samples are better. Preferably, the multiple different is less than 100. Using the equation of “standard method”, an activity value corresponding to each Nattokinase sample with one dilution multiple is calculated. As such, the sample curve can be depicted. For example, as shown in FIG. 5, there are nine Nattokinase samples with an interval multiple as 50. Therefore, nine Nattokinase samples are diluted by dulition multiples of 100, 150, 200, 250, 300, 350, 400, 450 and 500 to calculate the activity values of 5467 FU, 7200 FU, 12000 FU, 15600 FU, 17276 FU, 20800 FU, 21600 FU and 26600 FU respectively, and a sample curve 30 is obtained.

Further, in step 13, put the standard curve 20 with the sample curve 30 in one diagram, as shown in FIG. 5, and there will have an intersection P of the standard curve 20 and the sample 30, which is determined as the standard activity value of the indeterminate Nattokinase in step 14. For example, P has a value of about 12000 FU and is determined as the standard activity value of the indeterminate Nattokinase.

Accordingly, there is no need to purchase any other extra experiment equipment or increase any cost to determine an unknown activity value of Nattokinase by the present invention.

The above description is given by way of example, and not limitation. Given the above disclosure, one skilled in the art could devise variations that are within the scope and spirit of the invention disclosed herein, including configurations ways of the recessed portions and materials and/or designs of the attaching structures. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A method of determining a standard activity value of Nattokinase, comprising:
preparing a standard curve with respect to a plurality of Nattokinases having predetermined activity values by calculating corresponding dilution multiples using a standard essay;
obtaining a sample curve with respect to an indeterminate Nattokinase by diluting the indeterminate Nattokinase with a plurality sample dilution multiples to calculate corresponding activity values using the standard essay; and
determining a standard activity of the indeterminate Nattokinase by comparing the standard curve and the sample curve in a diagram to obtain an intersection.

2. The method as claimed in claim 1, wherein the standard essay includes comparing a Nattokinase sample to a dummy sample by using an equation of FU/g=(A.sub.τ−A.sub.B)/0.01.times.1/60.times.1/0.1.times.D, where the A.sub.τ and the A.sub.B are absorbances of the Nattokinase sample and the dummy sample respectively measured at a wavelength of 275 nm, to obtain a relation between the activity value per gram FU/g and the dilution multiple D.

3. The method as claimed in claim 1, wherein a multiple difference between the sample dilution multiples is less than 100.
